(19) United States
(12) Patent Application Publication
(10) Pub. No.: US 2018/0207019 A1
(43) Pub. Date: Jul. 26, 2018

(54) STOMAL IRRIGATION SUPPORT DEVICE

(71) Applicant: Myrtle Technologies, LLC, Lewiston, ID (US)

(72) Inventors: Jacob P. Kershisnik, III, Lewiston, ID (US); Adam Ethridge, Lewiston, ID (US); Pat Kershisnik, Lewiston, ID (US)

(73) Assignee: Myrtle Technologies, LLC, Lewiston, ID (US)

(21) Appl. No.: 15/872,882

(22) Filed: Jan. 16, 2018

Related U.S. Application Data
(60) Provisional application No. 62/446,237, filed on Jan. 13, 2017.

(51) Int. Cl. A61F 5/442; A61F 5/44

(52) U.S. Cl. CPC A61F 5/442; A61M 25/02; A61F 5/4405

(57) ABSTRACT

A stomal irrigation support device including an irrigation sleeve support and a waste receptacle stop is provided. The irrigation sleeve support can have a structural configuration to support an irrigation sleeve and to extend across a portion of a user's body and onto a waste receptacle during use. The waste receptacle stop can be coupled to the irrigation sleeve support and can be positioned to impede movement of the irrigation sleeve support toward the waste receptacle during use.

### CROSS-REFERENCE TO RELATED APPLICATIONS

This application claims the benefit of U.S. Provisional Patent Application Ser. No. 62/446,237 filed on Jan. 13, 2017, which is herein incorporated by reference in its entirety.

### BACKGROUND

Medical conditions affecting the gastrointestinal system can necessitate the placement of an artificial stoma. The stoma can be temporary or permanent, the placement of which can vary based on the medical condition and the physical characteristics of the individual. Stomas can be placed for drainage or for other purposes, and while specific aspects of care can vary depending on purpose and location, all bowel stomas can benefit from periodic irrigation.

### BRIEF DESCRIPTION OF THE DRAWINGS

For a fuller understanding of the nature and advantage of the present invention, reference is being made to the following detailed description of preferred embodiments and in connection with the accompanying drawings, in which:

FIG. 1 is a schematic drawing of a stoma irrigation support device in accordance with one aspect of the present disclosure;

FIG. 2 is a schematic drawing of a stoma irrigation support device in accordance with another aspect of the present disclosure;

FIG. 3A is a schematic drawing of an end view of a stoma irrigation support device in accordance with another aspect of the present disclosure;

FIG. 3B is a schematic drawing of an end view of a stoma irrigation support device in accordance with another aspect of the present disclosure;

FIG. 3C is a schematic drawing of an end view of a stoma irrigation support device in accordance with another aspect of the present disclosure;

FIG. 4 is a schematic drawing of a stoma irrigation support device in accordance with another aspect of the present disclosure;

FIG. 5 is a schematic drawing of a stoma irrigation support device in accordance with another aspect of the present disclosure;

FIG. 6 is a schematic drawing of a stoma irrigation support device in accordance with another aspect of the present disclosure;

FIG. 7 is a schematic drawing of a top view of a stoma irrigation support device in accordance with another aspect of the present disclosure;

FIG. 8a is a schematic drawing of a stoma irrigation support device in accordance with another aspect of the present disclosure;

FIG. 8b is a schematic drawing of a stoma irrigation support device in accordance with another aspect of the present disclosure; and

FIG. 9 is a schematic drawing of a stoma irrigation support device in accordance with another aspect of the present disclosure.

### DETAILED DESCRIPTION

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals, when appearing in different drawings, represent the same or similar element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, to provide a thorough understanding of various embodiments. One skilled in the relevant art will recognize, however, that such detailed embodiments do not limit the overall concepts articulated herein, but are merely representative thereof. One skilled in the relevant art will also recognize that the invention can be practiced without one or more of the specific details, or with other components, layouts, etc. In other instances, well-known structures, materials, or operations may not be shown or described in detail to avoid obscuring aspects of the disclosure.

In this application, “comprises,” “comprising,” “containing,” “having” and the like can have the meaning ascribed to them in U.S. Patent law and can mean “includes,” “including,” and the like, and are generally interpreted to be open-ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, “enhanced,” “improved,” “performance-enhanced,” “upgraded,” and the like, when used in connection with the description of a device or process, refers to a characteristic of the device or process that provides measurably better form or function as compared to previously known devices or processes. This applies both to the form and function of individual components in a device or process, as well as to such devices or processes as a whole.

As used herein, the term “substantially” refers to the complete or nearly complete extent or degree of an action, characteristic, property, state, structure, item, or result. For example, an object that is “substantially” treated with a UV coating would mean that the object is either completely treated or nearly completely treated with the UV coating. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” toxins would either completely lack toxins, or so nearly completely lack toxins that the effect would be the same as if it completely lacked toxins. In other words, a composition that is “substantially free of” an ingredient or element may still actually contain such item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Reference throughout this specification to “an example,” “an embodiment,” or “an aspect” means that a particular feature, structure, or characteristic described in connection with the example, embodiment, or aspect is included in at least one example, embodiment, or aspect. Thus, appearances of phrases including “an example,” “an embodiment,” or “an aspect” in various places throughout this specification are not necessarily all referring to the same example, embodiment, or aspect.

Objects and structures described herein as being “adjacent to” each other may be in physical contact with each other, in close proximity to each other, or in the same general region or area as each other, as appropriate for the context in which the phrase is used.

As used herein, “coupled” refers to a relationship of physical connection or attachment between one item and another item, and includes relationships of either direct or indirect connection or attachment. Any number of items can be coupled, such as materials, components, structures, layers, devices, objects, etc.

The terms “upwardly-facing surface,” “upward,” “downward,” and the like in the description and in the claims, if any, are used for descriptive purposes and not necessarily for describing permanent relative positions but rather the relationship to a stoma irrigation support device or a component thereof.

An initial overview of embodiments is provided below and specific embodiments are then described in further detail. This initial summary is intended to aid readers in understanding the disclosure more quickly, but is not intended to identify key or essential technological features, nor is it intended to limit the scope of the claimed subject matter.

### Example Embodiments

In one example of stomal irrigation, a liquid, such as water, saline, or the like, is flushed through the colon to facilitate the evacuation of wastes. The liquid, along with the wastes, drains through an irrigation sleeve from the stoma and into a waste receptacle. As used herein, a “waste receptacle” can refer to any receptacle capable of accepting wastes, including but not limited to, a toilet, a bucket, a bedpan, or the like. The irrigation process can take up to an hour or more, and can require the user, or someone assisting the user, to hold the outlet of the irrigation sleeve in position at the waste receptacle for long durations, even up to the entire duration of the irrigation process. This can be particularly challenging, as it requires the user to remain immobile for an extended period of time while holding the outlet of the irrigation sleeve in a position that allows proper drainage into the waste receptacle. This process can be especially challenging for users that are in wheelchairs, are bedridden, or are very young.

The present disclosure provides solutions to various problems, including those outlined above, which can assist users in managing many of the difficulties associated with traditional stomal irrigation. In one example, a stomal irrigation device (i.e. irrigation sleeve support) can overcome difficulties by providing support to an irrigation sleeve during the irrigation process, both at the user and over the waste receptacle. For example, an irrigation sleeve support can support the irrigation sleeve from the user to the waste receptacle, and a waste receptacle stop coupled thereto can maintain the irrigation sleeve support in a fixed position at the waste receptacle. In addition to supporting the irrigation sleeve, the irrigation sleeve support can be angled from the user to the waste receptacle, thus allowing liquid and waste in the irrigation sleeve to drain by gravity to the waste receptacle. The angle can be due to a height difference between structures upon which the irrigation sleeve support is resting, based on the structural configuration of the irrigation sleeve support, or a combination thereof.

Furthermore, the irrigation processes can be time-consuming, often lasting an hour or more, during which time the user maintains the irrigation sleeve over the waste receptacle. The irrigation sleeve support device can be beneficial to users that are in wheelchairs, that are bedridden, that may have difficulties in hand coordination, that are seated near the waste receptacle, and the like, because the user is not required to hold the irrigation sleeve in an uncomfortable, extended position for the entirety of the irrigation process. Therefore, the need to focus intently on the irrigation is greatly reduced. This additionally frees the user to focus on other tasks, more enjoyable pastimes, and the like during the irrigation process, which can greatly increase enjoyment and quality of life. Furthermore, the stoma irrigation support device can allow a user to sit near the waste receptacle in a chair, which can provide more comfort than sitting on a toilet for the entirety of the irrigation.

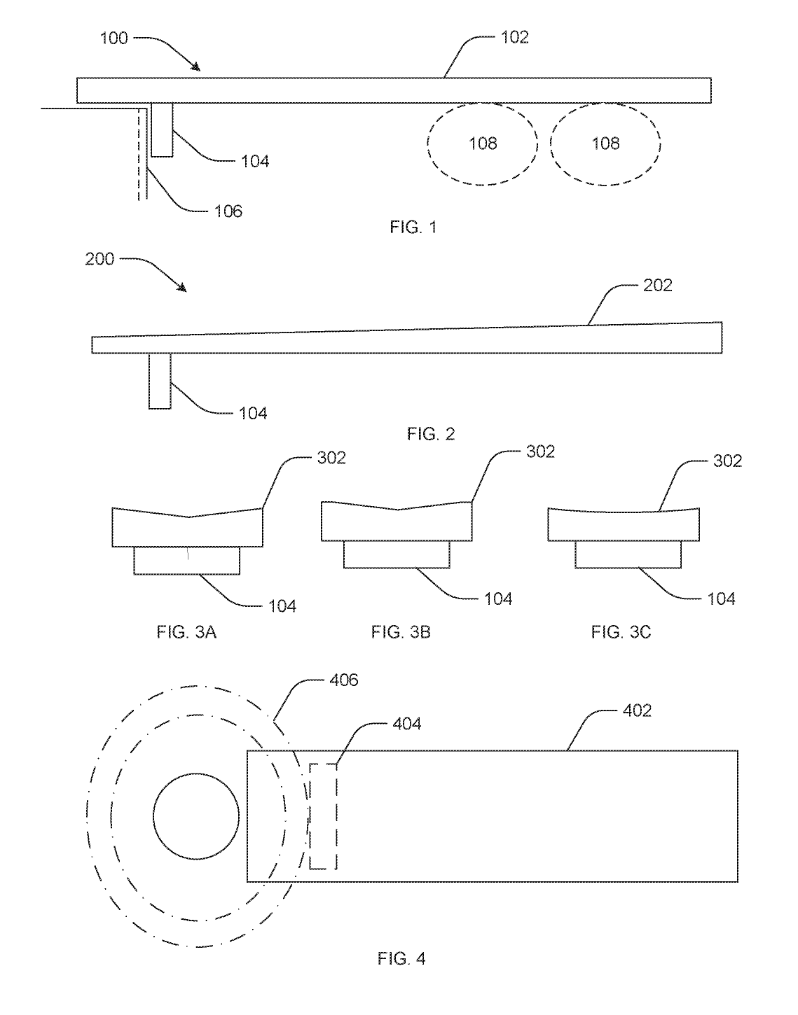

In one example, as shown in FIG. 1, a stomal irrigation support device 100 can include an irrigation sleeve support 102 having a structural configuration to support an irrigation sleeve and to extend from a user's stoma, across a portion of the user's body, and onto a waste receptacle 106 when in use. A waste receptacle stop 104 can be coupled to the irrigation sleeve support, and can be positioned to impede movement of the irrigation sleeve support 102 toward the waste receptacle 106 during use. In some examples, the waste receptacle stop 104 can maintain the irrigation sleeve support 102 in a fixed position at a waste receptacle 106. In such cases, one end of the irrigation sleeve support 102 can rest on or over the waste receptacle 106, and the other end can rest on any number of structures, depending on how the user is seated, standing, or lying, and what the user is seated, standing, or lying on. For example, as shown in FIG. 1, the irrigation sleeve support 102 can rest on a user's thighs 108. In other examples, the irrigation sleeve support 102 can rest on a user's waist, stomach, or the like. In yet other examples, the irrigation sleeve support 102 can rest on a wheelchair armrest, a chair armrest, a bed rail, a table, or the like. The waste receptacle stop 104 can extend downward from the irrigation sleeve support 102, and thus position the irrigation sleeve support 102 in a proper position over the waste receptacle 106, while at the same time providing a stop that is operable to prevent the irrigation sleeve support 102 from sliding away from the user and further over the waste receptacle 106. As such, the irrigation sleeve support 102 can provide a stable surface upon which an irrigation sleeve can rest, to allow hands-free stomal irrigation.

The irrigation sleeve support can be any size, shape, or geometry that is useful for assisting a user in an irrigation process. For example, the upper surface can be rectangular, ovoid, oblong, or the like. In some examples, an edge of the irrigation sleeve support can have a cutout to conform to a portion of a user's body, a structure upon which the user is sitting, a room structure, or the like. In some examples, the irrigation sleeve support can have a uniform thickness, while in other examples, the irrigation sleeve support can have a nonuniform thickness. As one example, as is shown in FIG. 2, a stomal irrigation support device 200 can have an irrigation sleeve support 202 that tapers from the end that is placed near the stoma to the opposite end that is placed at the waste receptacle. This configuration can facilitate the drainage of fluid and waste along the irrigation sleeve support 202. In yet other examples, the irrigation sleeve support can have an upwardly facing contoured surface. Such a surface can be structurally configured to receive and to stabilize the irrigation sleeve along the irrigation sleeve support during use. For example, the upwardly facing surface can be thicker along the sides and thinner near the center of the irrigation sleeve support. The contoured surface can extend along a portion or the entire length of the irrigation sleeve support, and can be symmetrical or asymmetrical along the width or the length. End views of exemplary irrigation sleeve supports 302 having example contoured surfaces are shown in FIGS. 3A-3C. The contoured surface can facilitate positioning the irrigation sleeve along the irrigation sleeve support and provide added stability during the irrigation process. The contoured surface can additionally contain any accidental spills and facilitate cleanup by directing cleansing fluids toward the waste receptacle.

The waste receptacle stop can be of any useful shape or size, and can be positioned to impede the movement of the irrigation sleeve device toward the waste receptacle during use. The waste receptacle stop can be formed as an extension of the irrigation sleeve support, or the waste receptacle stop can be a separately formed structure that is subsequently attached to the irrigation sleeve support. The waste receptacle stop can extend downwardly from the irrigation sleeve support in a position that abuts the waste receptacle as the irritation sleeve support rests thereon. In yet other examples, the waste receptacle stop can extend from a side of the irrigation sleeve support. The waste receptacle stop can be configured in a variety of shapes, sizes, and thicknesses, and is not considered to be limiting.

In one example, the waste receptacle stop can be positioned to impede movement of the irrigation sleeve support toward the waste receptacle during use. In some instances, a user can assist in the draining of liquid and wastes from the irrigation sleeve by pressing and/or pushing on the irrigation sleeve from the stoma end towards the waste receptacle. The force applied by the user presses the waste receptacle stop against the waste receptacle, thus further stabilizing the irrigation sleeve support. In one example as shown in FIG. 4, an irrigation sleeve support 402 can include a waste support stop 404 positioned to abut the side of a waste receptacle 406, indicated by a dashed line. In this example embodiment, the irrigation sleeve support 402 extends onto the waste receptacle 406.

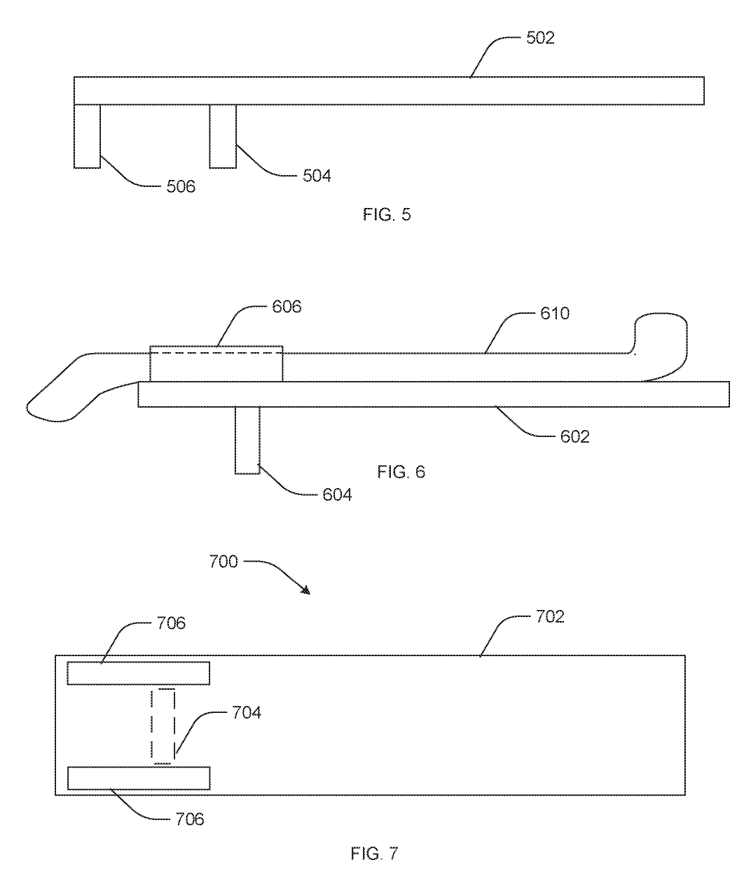

In other examples, the stoma irrigation support device can include multiple waste receptacle stops. As shown in FIG. 5, for example, an irrigation sleeve support 502 can include a first waste receptacle stop 504 and a second waste receptacle stop 506. In this case, the first waste receptacle stop 504 can be positioned to impede movement of the irrigation sleeve support toward the waste receptacle, and the second waste receptacle stop 506 can be positioned to impede movement of the irrigation sleeve support toward the user (i.e., away from the waste receptacle). Depending on the design configuration, in one example the waste receptacle stops can be positioned to abut either side of a wall of the waste receptacle, on opposite inside walls of the waste receptacle, or on opposite outside walls of the waste receptacle. In some cases where the irrigation sleeve support extends across the opening of the waste receptacle, an opening can be provided in the irrigation sleeve support to receive the end of the irrigation sleeve to allow contents to empty therethrough.

In some examples, the stomal irrigation support device can further include an irrigation sleeve stop coupled to the irrigation sleeve support in a position to impede an irrigation sleeve from falling off the irrigation sleeve support during use. The irrigation sleeve stop can be a structure, a fastener, or a combination thereof. An irrigation sleeve stop structure can provide a surface against which the irrigation sleeve can rest or otherwise bump into, thus preventing the irrigation sleeve from falling off the irrigation sleeve support. A fastener can function as an irrigation sleeve stop by loosely securing the irrigation sleeve to the irrigation sleeve support such that the irrigation sleeve does not fall therefrom.

An exemplary irrigation sleeve stop is shown in FIG. 6. The stomal irrigation support device can include an irrigation sleeve support 602, a waste receptacle stop 604, and an irrigation sleeve stop 606. The irrigation sleeve stop 606 in this example is positioned at the waste receptacle-end of the irrigation sleeve support 602. In some examples, the irrigation sleeve stop 606 can assist in positioning the irrigation sleeve 610 over the waste receptacle. In yet other examples, the irrigation sleeve stop 606 can maintain the position of the irrigation sleeve to a degree that allows the user to more fully utilize their hands to press liquid and wastes through the irrigation sleeve, or to free their hands for other tasks, either related or unrelated to the irrigation process. In some examples, the irrigation sleeve stop can be positioned at one end of the irrigation sleeve support. In other examples, the irrigation sleeve stop can extend across a portion or all of the irrigation sleeve support. In yet another example, the irrigation sleeve stop can be in multiple discrete locations along the irrigation sleeve support 602.

FIG. 7 shows a top view of one example embodiment of a stomal irrigation support device 700 with two irrigation sleeve stops 706 positioned along portions of the sides of the irrigation sleeve support 702. The irrigation sleeve stop(s) 706 can be positioned at either or both sides of the irrigation sleeve support. Also shown in FIG. 7 is a waste receptacle stop 704, which is located on the bottom surface of the irrigation sleeve support 702 from the irrigation sleeve stop 706.

In one example, the irrigation sleeve support can be divided into two support portions along a breakpoint such that each support portion has breakpoint end, where the breakpoint is configured to reversibly reduce the length of the irrigation sleeve support. Additionally, a breakpoint support can be coupled across the breakpoint ends to provide support to the irrigation sleeve support during use. In one example, the breakpoint support can include a channel configured to allow the support portions to slide past one another at the breakpoint ends in order to reduce the length of the irrigation sleeve support. The breakpoint support can coupled to the irrigation sleeve support such that the support portions are permanently coupled together, or the breakpoint support can coupled to the irrigation sleeve support such that the support portions are reversibly coupled together during use.

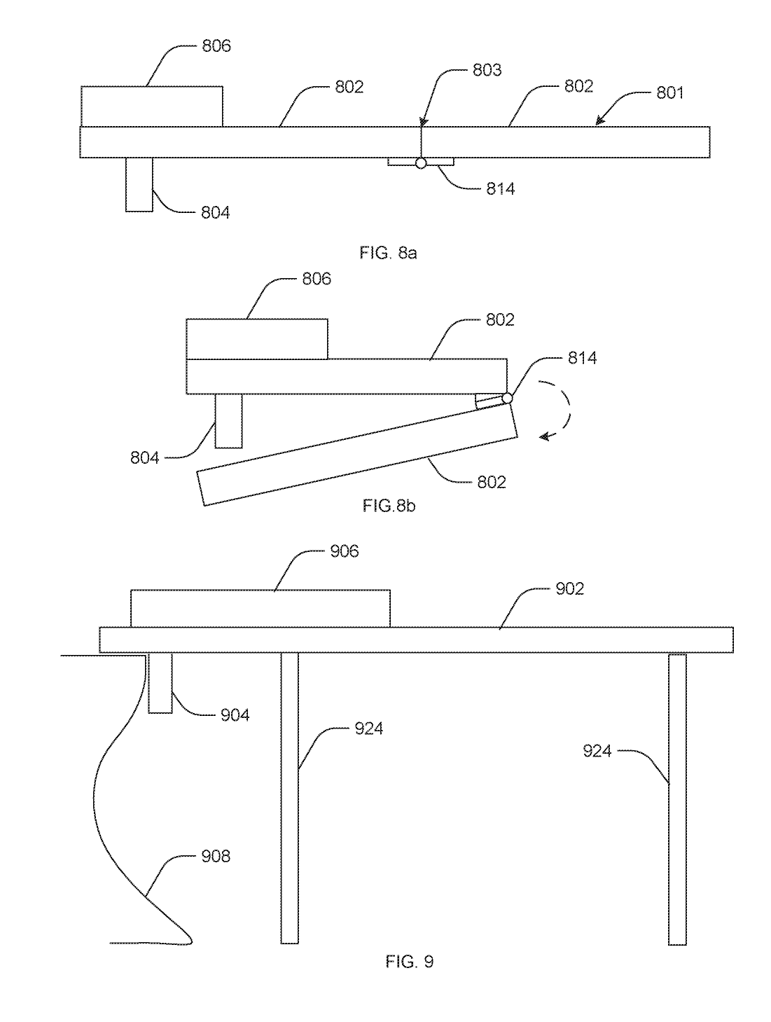

In one example, the irrigation sleeve support can further include a hinge coupled across the breakpoint, either as the breakpoint support or in addition to a separate breakpoint support. As shown in the example of FIGS. 8a,b, an irrigation sleeve support 801 includes support portions 802, which are coupled together by a hinge 814. The irrigation sleeve support can include a waste receptacle stop 804 and an irrigation sleeve stop 806. The hinge 814 is positioned and operable to facilitate folding of the irrigation sleeve support 801 at the breakpoint 803, thus reducing the overall length of the stomal irrigation support device.

In another example, the stomal irrigation support device can include a pad or cushion coupled to an underside portion of the irrigation sleeve support to rest on the user in order to provide cushioning between the user and the irrigation sleeve support. In a further example, the stomal irrigation support device can include a decorative feature. In some cases, the decorative feature can be interchangeable and/or can have a functional purpose. For example, the decorative feature can be incorporated into the irrigation sleeve stop or a portion thereof. In yet other examples, the decorative feature can be purely aesthetic. The incorporation of the decorative feature can assist the user's mental health and/or enjoyment during use. A decorative feature can be desirable in stoma irrigation support devices intended for use in pediatric care.

In a further example, the irrigation sleeve support can include a stabilizer coupled to the irrigation sleeve support and configured to contact a floor during use. A stabilizer can be used to assist in providing support anywhere along the stomal irrigation support device. In one example, a stabilizer can be used to provide added support near the waste receptacle. In another example, as is shown in FIG. 9, stabilizers 924 can be used near both ends of the irrigation sleeve support 902. In some examples, stabilizers 924 can include two pairs of legs to allow the irrigation sleeve support 902 to be self-standing. In some examples, the stabilizer can be detachable. In yet other examples, the stabilizer can include a hinge such that the stabilizer can be folded for compact storage, and/or can have a vertical adjustment mechanism that allows the length of the stabilizer to be adjusted. For example, the stabilizer could be telescoping and/or folding. Also shown in FIG. 9 is a waste receptacle stop 904 contacting a waste receptacle 908 and an irrigation sleeve stop 906.

In yet another example, the stoma irrigation support device can include a user fastening mechanism to secure the stoma irrigation support device to the user, or to a location where the user is located. In one example, the user fastening mechanism can be a belt, a lanyard, or a combination thereof. The user fastening mechanism can attach to the irrigation sleeve support. A user fastening mechanism can ensure that the stoma irrigation support device does not become separated from the user during the irrigation process, thereby allowing a user to move freely during irrigation.

In a further example, the stoma irrigation support device can include a protective covering. The protective covering can be a protective sleeve, protective liner, other protective material, or the like, that can be applied to the irrigation sleeve support, the waste receptacle stop, the irrigation sleeve stop, or any other feature of the stoma irrigation support device prior to use. The protective covering can shield the stoma irrigation support device from accidental contact with waste materials, thus allowing for easy cleanup following irrigation. The protective covering can be reusable or disposable. In one example, the protective covering can cover or enclose a substantial portion of the stoma irrigation support device. In yet another example, the protective covering can cover or enclose at least a portion of the stoma irrigation support device.

The protective covering can include a tacky surface which can assist in securing the protective covering to the stoma irrigation support device, an irrigation sleeve to the stoma irrigation support device, the waste receptacle stop to the waste receptacle, or any combination thereof. In some examples, the protective covering can be made from a textured material that can increase friction to further immobilize the irrigation sleeve support and/or the irrigation sleeve. In some examples, the protective covering can be made of a flushable material.

In yet another example, the irrigation sleeve support can include an accessory holder. The accessory holder can allow for the attachment or provide a receptacle to receive a sprayer, wipe (i.e. personal care wipes and/or cleaning/disinfecting wipes), sanitizing solution, irrigation sleeve, electronic device, reading material, or the like. The shape, size, and location of the accessory holder can vary depending on the desired use.

The stomal irrigation support device described above can be made from a variety of materials, and any materials capable of forming the structures of the present disclosure are included in the present scope. Non-limiting examples can include polymeric materials, metals, woods, synthetic or natural rubbers, fabrics, and the like. The material of the stoma irrigation support device can also be treated to impart various properties to the material. For example, the coating can be an antimicrobial coating, UV resistance coating, or the like. The irrigation sleeve support can be flexible or rigid, depending on the design of the device.

Further presented herein is a stomal irrigation support system. The system can include a stoma irrigation support device and an irrigation sleeve. The stoma irrigation support device can include an irrigation sleeve support and a waste receptacle stop as described above. The device can further include any combination of design features.

Further presented herein is a method of supporting an irrigation sleeve for a stomal irrigation procedure, including positioning a stomal end of an irrigation sleeve support near a stoma of a user, such that the irrigation sleeve support extends across a portion of the user's body, positioning a waste receptacle-end of the irrigation sleeve support at a waste receptacle, and positioning an irrigation sleeve on the irrigation sleeve support to extend from the stoma of the user to the waste receptacle.

The described features, steps, or characteristics may be combined in any suitable manner in one or more embodiments. It will be apparent to those of ordinary skill in the art that numerous modifications in form, usage and details of implementation can be made without the exercise of inventive faculty, and without departing from the principles and concepts of the invention presented herein.

What is claimed:

1. A stomal irrigation support device, comprising:
an irrigation sleeve support having a structural configuration to support an irrigation sleeve, and to extend from a user's stoma, across a portion of the user's body, and onto a waste receptacle when in use; and
a waste receptacle stop coupled to the irrigation sleeve support, the waste receptacle stop positioned to impede movement of the irrigation sleeve support toward the waste receptacle during use.

2. The device of claim 1, wherein the irrigation sleeve support is structurally configured to be sufficiently stable when in use to allow hands-free stomal irrigation.

3. The device of claim 1, wherein to extend from the user's stoma, across the portion of the user's body, and onto the waste receptacle when in use, the irrigation sleeve support is structurally configured to be supported on the portion of the user's body and on the waste receptacle.

4. The device of claim 1, wherein to extend from the user's stoma, across the portion of the user's body, and onto the waste receptacle when in use, the irrigation sleeve support is structurally configured to be supported on a structure upon which the user is seated and on the waste receptacle.

5. The device of claim 1, wherein the waste receptacle stop is further configured to impede movement of the irrigation sleeve support toward the user during use.

6. The device of claim 1, further comprising an irrigation sleeve stop coupled to the irrigation sleeve support in a position to impede the irrigation sleeve from falling off the irrigation sleeve support during use.

7. The device of claim 6, wherein the irrigation sleeve stop extends away from an upwardly-facing surface of the irrigation sleeve support.

8. The device of claim 1, wherein the irrigation sleeve support further comprises a contoured surface structurally configured to receive and to stabilize the irrigation sleeve along the irrigation sleeve support during use.

9. The device of claim 1, wherein the irrigation sleeve support further comprises two support portions divided along a breakpoint such that each support portion has a breakpoint end, wherein the breakpoint is positioned to reversibly reduce the length of the irrigation sleeve support.

10. The device of claim 9, further comprising a breakpoint support coupled across the breakpoint ends.

11. The device of claim 10, wherein the breakpoint support comprises a channel configured to allow the support portions to slide past one another at the breakpoint ends to reduce the length of the irrigation sleeve support.

12. The device of claim 10, wherein the breakpoint support secures the two portions of the support together at the breakpoint ends.

13. The device of claim 12, wherein the breakpoint support is a hinge configured to allow folding of the support at the breakpoint.

14. The device of claim 10, wherein the breakpoint support reversibly secures the two portions of the support together at the breakpoint ends.

15. The device of claim 1, further comprising a stabilizer coupled to the irrigation sleeve support and configured to contact a floor during use.

16. A stomal irrigation support system, comprising:
the stomal irrigation support device of claim 1; and
an irrigation sleeve.

17. A method of supporting an irrigation sleeve for a stomal irrigation procedure, comprising;
positioning a stomal end of an irrigation sleeve support near a stoma of a user, such that the irrigation sleeve support extends across a portion of the user's body;
positioning a waste receptacle-end of the irrigation sleeve support at a waste receptacle; and
positioning an irrigation sleeve on the irrigation sleeve support to extend from the stoma of the user to the waste receptacle.

18. The method of claim 17, wherein the waste receptacle-end of the irrigation sleeve support is supported on the waste receptacle.

19. The method of claim 17, wherein the irrigation sleeve support extends across the portion of the user's body further comprises the irrigation sleeve support is supported on the portion of the user's body.

20. The method of claim 19, wherein the portion of the user's body comprises thighs, waist, or stomach.
